(19) United States
(12) Patent Application Publication
(10) Pub. No.: US 2010/0197628 A1
(43) Pub. Date: Aug. 5, 2010

(54) METHODS OF TREATING PSYCHIATRIC, SUBSTANCE ABUSE, AND OTHER DISORDERS USING COMBINATIONS CONTAINING OMEGA-3 FATTY ACIDS

(76) Inventors: Perry F. Renshaw, Bedford, MA (US); William A. Carlezon, JR., Belmont, MA (US); Bruce M. Cohen, Lexington, MA (US)

Correspondence Address: CLARK & ELBING LLP, 101 FEDERAL STREET, BOSTON, MA, 02110, US

(21) Appl. No.: 12/704,964

(22) Filed: Feb. 12, 2010

Related U.S. Application Data
(63) Continuation of application No. 10/962,096, filed on Oct. 8, 2004, now abandoned.
(60) Provisional application No. 60/509,714, filed on Oct. 8, 2003.

(51) Int. Cl. A61K 31/7068; A61P 25/24; A61P 25/00

(52) U.S. Cl. 514/49

(57) ABSTRACT

The invention provides methods for treating or preventing psychiatric disorders, substance abuse disorders, and other conditions, e.g., cardiovascular disease and cancer, involving administration of a therapeutically-effective amount of a cytosine-containing or cytidine-containing compound, creatine-containing compound, adenosine-containing, adenosine-elevating compound, omega-3 fatty acids, or combinations thereof to a mammal. The invention further provides methods of enhancing neurodevelopment and delaying premature pregnancy by administration of an effective amount of a cytosine-containing or cytidine-containing compound, creatine-containing compound, adenosine-containing, adenosine-elevating compound, omega-3 fatty acids, or combinations thereof to a mammal.

### CROSS-REFERENCE TO RELATED APPLICATIONS

This application is a continuation of U.S. application Ser. No. 10/962,096, filed Oct. 8, 2004, which claims benefit of U.S. Provisional Application No. 60/509,714, filed Oct. 8, 2003, each of which is hereby incorporated by reference.

### STATEMENT AS TO FEDERALLY SPONSORED RESEARCH

This invention was funded, in part, by grants DA09448, DA11321, and DA09448, from the National Institute on Drug Abuse, and MH48343, MH53636, and MH63266 from the National Institute of Mental Health. The government may have certain rights in the invention.

### BACKGROUND OF THE INVENTION

This invention relates to compositions and methods for the treatment of psychiatric, e.g., depressive, substance abuse, or other disorders.

Psychiatric and substance abuse disorders present unique complications for patients, clinicians, and care givers. These disorders are difficult to diagnose unequivocally and fear of societal condemnation, as well as lack of simple and effective therapies, often results in patients who are reluctant to disclose their symptoms to health professionals, leading to adverse societal and health consequences.

Psychiatric and substance abuse disorders include alcohol and opiate abuse or dependence, depression, dysthymia, and attention-deficit hyperactivity disorder, among others, and occur in people of all ages and backgrounds.

Use of substances such as alcohol and opiates often leads to addiction and dependence on these substances, causing a variety of adverse consequences, including clinical toxicity, tissue damage, physical dependence and withdrawal symptoms, and an impaired ability to maintain social and professional relationships. The etiology of substance abuse or dependence is unknown, although factors such as the user's physical characteristics (e.g., genetic predisposition, age, or weight), personality, or socioeconomic class have been postulated to be determinants.

Depression and dysthymia are prevalent disorders that are often chronic and associated with frequent relapses and long duration of episodes. These disorders include psychosocial and physical impairment and a high suicide rate among those affected. A lifetime prevalence of approximately 17% has been widely reported, and the likelihood of recurrence is more than 50% (Angst, J. Clin. Psychiatry 60 Suppl. 6:5-9, 1999). Because most antidepressants with clinical efficacy act upon monoamines (primarily norepinephrine and serotonin), much research on depression has focused upon interactions between these neurotransmitters and their reuptake transporters and receptor proteins.

Most pharmacotherapies for depression require weeks or months of treatment despite immediate effects on brain monoamine transmission. As a result, research has become progressively less focused upon receptors themselves and more focused upon the intracellular mechanisms of antidepressant treatments. The neurological mechanisms underlying depression and dysthymia are poorly understood, with a concomitant lack of suitable pharmacological therapies for the treatment of these disorders. Current therapies often have many adverse effects and are not suitable for administration to certain cohorts. For example, depression in the elderly, particularly in those in long-term care facilities, is common and is often more refractory to treatment than depression in young or middle-aged adults; however, the elderly are particularly sensitive to the common adverse effects of many antidepressant drugs, particularly the anticholinergic side effects. Similarly, therapies that are suitable for administration to adults may not be suitable for children.

Attention-deficit hyperactivity disorder (ADHD) is a highly heritable and prevalent neuropsychiatric disorder estimated to affect 6% of the school-age children in the United States. ADHD typically occurs in early childhood and persists into adulthood, but is often not diagnosed until or after adolescence. Clinical hallmarks of ADHD are inattention, hyperactivity, and impulsivity, which often respond to treatment with stimulants (e.g., methylphenidate, dextroamphetamine, or magnesium pemoline), although non-stimulant drugs such as beta-blockers (e.g., propranolol or nadolol), tricyclic antidepressants (e.g., desipramine), and anti-hypertensives (e.g., clonidine) are also used. Treatment with these drugs, however, is complicated by adverse effects, including the possibility of abuse of the medication, growth retardation, disturbance of heart rhythms, elevated blood pressure, drowsiness, depression, sleep disturbances, headache, stomachache, appetite suppression, rebound reactions, and by the unclear long-term effects of drug administration on brain function.

Simple and effective pharmacological treatments for these disorders have proven scarce to date. It would be beneficial to provide pharmacotherapies suitable for administration to all populations, including the elderly and children, for the treatment of substance abuse and psychiatric disorders, such as depression.

### SUMMARY OF THE INVENTION

In general, the invention features methods of treating psychiatric disorders, substance abuse or dependency, and other disorders, and their symptoms, by administering a cytidine-containing, cytosine-containing, creatine-containing, uridine-containing, adenosine-containing, or adenosine-elevating compound, in combination with an omega-3 fatty acid to a mammal. Substance abuse and dependencies treated by the methods described herein include, for example, alcohol, opiate, cocaine, amphetamines, methamphetamine, and methylphenidate abuse or dependence. Psychiatric disorders treated by the methods described herein include mood disorders (e.g., unipolar depression, dysthymia, cyclothymia, and bipolar disorder), attention-deficit hyperactivity disorder (ADHD), anxiety disorders (e.g., panic disorder and generalized anxiety disorder), obsessive-compulsive disorder (OCD), post-traumatic stress disorder (PTSD), phobias, and psychotic disorders (e.g., schizophrenia and schizoaffective disorder). Preferred psychiatric disorders include unipolar depression, dysthymia, cyclothymia, panic disorder, generalized anxiety disorder, obsessive-compulsive disorder (OCD), post-traumatic stress disorder (PTSD), and phobias. Other disorders treated by the methods of the invention include cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, and gestational diabetes. In addition, the invention features methods of enhancing neurodevelopment and delaying premature birth by administering a cytidine-containing, cytosine-containing, creatine-containing, uridine-containing, adenosine-containing, adenosine-elevating compound, or omega-3 fatty acid to a mammal.

Any of the cytidine-containing, cytosine-containing, creatine-containing, uridine-containing, adenosine-containing, adenosine-elevating compounds, or omega-3 fatty acids of the invention may be administered separately or in combination. When a combination of compounds is employed, one or more of the compounds may be employed in a subtherapeutically effective amount or an amount insufficient alone to effect the desired outcome. In this embodiment, the combination is administered in a therapeutically effective amount or an amount sufficient to effect the desired outcome, even though one or more of the active ingredients is administered at less than an effective level. An exemplary combination for use in any of the methods described herein includes an omega-3 fatty acid and either a uridine-containing compound, a cytidine-containing compound, or a cytosine-containing compound.

The invention therefore further features compositions including a combination of an omega-3 fatty acid and either a uridine-containing compound, a cytidine-containing compound, or a cytosine-containing compound, e.g., wherein at least one compound is present in a subtherapeutically effective amount.

In preferred embodiments of any aspect of the invention, the cytidine-containing compound is cytidine, CDP, or CDP-choline; the cytidine-containing compound includes choline; and the mammal is a human child, adolescent, adult, or older adult. In other preferred embodiments, the CDP-choline is administered orally, and the administration is chronic.

The uridine-containing compound is for example uridine, UMP, UDP, UTP, or triacetyl uridine. Exemplary omega-3 fatty acids include eicosapentaenoic acid, docosahexaenoic acid, and α-linolenic acid, e.g., from fish oil, flaxseed oil, or microalgae.

In other preferred embodiments, a brain phospholipid (e.g., lecithin) or a brain phospholipid precursor (e.g., a fatty acid or a lipid), is also administered to the mammal. In other preferred embodiments, an antidepressant is also administered to the mammal.

In other preferred embodiments, the mammal has a co-morbid neurological disease, for example, post-stroke depression.

Treatment methods may also include a diagnosis of the particular disorder or condition by a physician or other medical professional prior to administration of the particular disorder or condition. Administration of the therapeutic compounds may also occur under the continuing care of a physician or medical professional.

As used herein, by “alcohol” is meant a substance containing ethyl alcohol. By “opiate” is meant any preparation or derivative of opium, which is a naturally occurring substance extracted from the seed pod of a poppy plant (e.g., Papaver somniferum) and which contains at least one of a number of alkaloids including morphine, noscapine, codeine, papaverine, or thebaine. Heroin, an illegal, highly addictive drug is processed from morphine. For the purposes of this invention, the term opiate includes opioids.

By “opioid” is meant a synthetic narcotic that resembles an opiate in action, but is not derived from opium.

By “abuse” is meant excessive use of a substance, particularly one that may modify body functions, such as alcohol or opiates.

By “dependency” is meant any form of behavior that indicates an altered or reduced ability to make decisions resulting, at least in part, from the use of a substance. Representative forms of dependency behavior may take the form of antisocial, inappropriate, or illegal behavior and include those behaviors directed at the desire, planning, acquiring, and use of a substance. This term also includes the psychic craving for a substance that may or may not be accompanied by a physiological dependency, as well as a state in which there is a compulsion to take a substance, either continuously or periodically, in order to experience its psychic effects or to avoid the discomfort of its absence. Forms of “dependency” include habituation, that is, an emotional or psychological dependence on a substance to obtain relief from tension and emotional discomfort; tolerance, that is, the progressive need for increasing doses to achieve and sustain a desired effect; addiction, that is, physical or physiological dependence which is beyond voluntary control; and use of a substance to prevent withdrawal symptoms. Dependency may be influenced by a number of factors, including physical characteristics of the user (e.g., genetic predisposition, age, gender, or weight), personality, or socioeconomic class.

By “dysthymia” or “dysthymic disorder” is meant a chronically depressed mood that occurs for most of the day, more days than not, for at least two years. In children and adolescents, the mood may be irritable rather than depressed, and the required minimum duration is one year. During the two year period (one year for children or adolescents), any symptom-free intervals last no longer than 2 months. During periods of depressed mood, at least two of the following additional symptoms are present: poor appetite or overeating, insomnia or hypersomnia, low energy or fatigue, low self-esteem, poor concentration or difficulty making decisions, and feelings of hopelessness. The symptoms cause clinically significant distress or impairment in social, occupational (or academic), or other important areas of functioning. The diagnosis of dysthymia is not made if: the individual has ever had a manic episode, a mixed episode, a hypomanic episode; has ever met the criteria for a cyclothymic disorder; the depressive symptoms occur exclusively during the course of a chronic psychotic disorder (e.g., schizophrenia); or if the disturbance is due to the direct physiological effects of a substance or a general medical condition. After the initial two-years of dysthymic disorder, major depressive episodes may be superimposed on the dysthymic disorder (“double depression”). (Diagnostic and Statistical Manual of Mental Disorders (DSM IV), American Psychiatric Press, 4^{th} Edition, 1994).

By “unipolar depression” or “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, and stroke). Generally, unipolar depression is more severe than dysthymia.

The essential feature of a major depressive episode is a period of at least two weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's preepisode status. The symptoms must persist for most of the day, nearly every day, for at least two consecutive weeks, and the episode must be accompanied by clinically significant distress or impairment in social, occupational (or academic), or other important areas of functioning. (Diagnostic and Statistical Manual of Mental Disorders (DSM IV), American Psychiatric Press, 4^{th} Edition, 1994).

By “neurological disease” is meant a disease, which involves the neuronal cells of the nervous system. Specifically included are prion diseases (e.g., Creutzfeldt-Jakob disease); pathologies of the developing brain (e.g., congenital defects in amino acid metabolism, such as argininosuccinicaciduria, cystathioninuria, histidinemia, homocystinuria, hyperammonemia, phenylketonuria, tyrosinemia, and fragile X syndrome); pathologies of the mature brain (e.g., neurofibromatosis, Huntington's disease, depression, amyotrophic lateral sclerosis, multiple sclerosis); conditions that strike in adulthood (e.g. Alzheimer's disease, Creutzfeldt-Jakob disease, Lewy body disease, Parkinson's disease, Pick's disease); and other pathologies of the brain (e.g., brain mishaps, brain injury, coma, infections by various agents, dietary deficiencies, stroke, multiple infarct dementia, and cardiovascular accidents). By “co-morbid” or “co-morbidity” is meant a concomitant but unrelated pathology, disease, or disorder. The term co-morbid usually indicates the coexistence of two or more disease processes.

By “attention-deficit hyperactivity disorder” or “ADHD” is meant a behavioral disorder characterized by a persistent and frequent pattern of developmentally inappropriate inattention, impulsivity, and hyperactivity. Indications of ADHD include lack of motor coordination, perceptual-motor dysfunctions, EEG abnormalities, emotional lability, opposition, anxiety, aggressiveness, low frustration tolerance, poor social skills and peer relationships, sleep disturbances, dysphoria, and mood swings (“Attention Deficit Disorder,” The Merck Manual of Diagnosis and Therapy (17^{th} Ed.), eds. M. H. Beers and R. Berkow, Eds., 1999, Whitehouse Station, N.J.).

By “treating” is meant the medical management of a patient with the intent that a cure, amelioration, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

By “therapeutically-effective amount” is meant an amount of a cytidine-containing, cytosine-containing compound, a uridine-containing compound, a creatine-containing compound, an adenosine-containing compound, an adenosine-elevating compound, an omega-3 fatty acid, or combination thereof sufficient to produce a healing, curative, prophylactic, stabilizing, or ameliorative effect in a particular treatment.

By “subtherapeutically-effective amount” is meant an amount of a cytidine-containing, cytosine-containing compound, a uridine-containing compound, a creatine-containing compound, an adenosine-containing compound, an adenosine-elevating compound, or omega-3 fatty acid not sufficient on its own to produce a healing, curative, prophylactic, stabilizing, or ameliorative effect in a particular treatment.

By “cytidine-containing compound” is meant any compound that includes, as a component, cytidine, CMP, CDP, CTP, dCMP, dCDP, or dCTP. Cytidine-containing compounds can include analogs of cytidine. Preferred cytidine-containing compounds include, without limitation, CDP-choline and cytidine 5′-diphosphocholine, frequently prepared as cytidine 5′-diphosphocholine [sodium salt] and also known as citicoline.

By “cytosine-containing compound” is meant any compound that includes, as a component, cytosine. Cytosine-containing compounds can include analogs of cytosine.

By “adenosine-containing compound” is meant any compound that includes, as a component, adenosine. Adenosine-containing compounds can include analogs of adenosine.

By “adenosine-elevating compound” is meant any compound that elevates brain adenosine levels, for example, compounds which inhibit or alter adenosine transport or metabolism (e.g., dipyridamole or S-adenosylmethionine).

By “uridine-containing compound” is meant any compound that includes as a component, uridine or UTP. Uridine-containing compounds can include analogs of uridine, for example, triacetyl uridine.

By “creatine-containing compound” is meant any compound that includes as a component, creatine. Creatine-containing compounds can include analogs of creatine.

By “phospholipid” is meant a lipid containing phosphorus, e.g., phosphatidic acids (e.g., lecithin), phosphoglycerides, sphingomyelin, and plasmalogens. By “phospholipid precursor” is meant a substance that is built into a phospholipid during synthesis of the phospholipid, e.g., fatty acids, glycerol, or sphingosine.

By “omega-3 fatty acid” is meant a fatty acid having an unsaturated bond three carbons from the omega carbon. This term encompasses the free acid, a salt, or an esterified form, e.g., a phospholipid. Omega-3 fatty acids may be mono- or polyunsaturated.

By “child or adolescent” is meant an individual who has not attained complete growth and maturity. Generally, a child or adolescent is under twenty-one years of age.

By “older adult” is meant an individual who is in the later stage of life. Generally, an older adult is over sixty years of age.

Unless otherwise stated, all psychiatric and substance abuse disorders are those described in Diagnostic and Statistical Manual of Mental Disorders, 4th ed., Text Revision, Washington, D.C.: American Psychiatric Association, 2000, hereby incorporated by reference.

The present invention provides therapeutics for substance abuse or dependencies, psychiatric disorders, and other disorders and conditions. The compounds utilized herein are relatively non-toxic, and CDP-choline, uridine, triacetyl uridine, and omega-3 fatty acids in particular, are pharmocokinetically understood and known to be well tolerated by mammals. The present invention, therefore, provides treatments that are likely to have few adverse effects and may be administered to children and adolescents, as well as the elderly, or those whose health is compromised due to existing physical conditions.

### BRIEF DESCRIPTION OF THE DRAWINGS

FIG. 1 is a bar graph showing the relative efficacies of CDP-choline and fluoxetine.

FIG. 2 is a graph showing phosphorus-31 MRS data from the human brain.

FIG. 3A is a T1 weighted anatomical image of the basal ganglia and thalamus, indicating regions of interest, used to sample the T2 relaxation times, for C (caudate), P (putamen), and T (thalamus).

FIG. 3B is a scatter plot of individual T2 relaxation times for the right putamen of ADHD children treated with placebo and of healthy children. The increased T2 relaxation times seen in the ADHD sample indicate diminished regional blood volume.

FIG. 4A is a graph showing the association between T2-RT in right putamen and accuracy on the performance of the computerized attention task for children with ADHD on placebo (closed circles) and normal controls (open circles). As indicated there is a significant inverse linear correlation between accuracy and T2 relaxation time (higher levels of T2-RT indicate lower perfusion).

FIG. 4B is a graph showing the percent change in T2-RT in the right putamen following treatment with methylphenidate in children with ADHD. Note that the degree of response is affected by the baseline level of activity. The higher the temporal scaling the greater the activity of the subject. T2-RT change values below zero indicate enhanced regional blood volume following methylphenidate administration.

FIG. 5 is a schematic illustration of the molecular structure of CDP-choline.

FIGS. 6A-6C are graphs showing the effects of the standard antidepressant drugs using two separate but complementary methods of scoring. (A) When latency to become immobile (Mean±SEM) was measured, desipramine (DMI), fluoxetine (FLX) and citalopram (CIT) increased latencies to become immobile. (B) When behavioral sampling was used, DMI caused decreases in occurrences of immobility and increases in occurrences of climbing, without affecting occurrences of swimming (Means±SEM). This pattern of behaviors is consistent with a noradrenergic mechanism of action (Detke et al. Psychopharmacology 121:66-72 1995). In contrast, FLX and CIT decreased immobility and increased swimming without affecting climbing, a pattern of behaviors consistent with a serotonergic mechanism of action (Detke et al. Psychopharmacology 121:66-72 1995). (C) The antidepressant drugs did not affect the weights of the rats. *P <0.05, **P<0.01, Fisher's HSD tests, 7-12 rats per group.

FIGS. 7A-7C are graphs showing the effects of uridine (URI) alone on behaviors in the FST. (A) URI dose-dependently increased latencies to become immobile. (B) URI dose-dependently decreased immobility and increased swimming without affecting climbing, a pattern of behaviors similar to that seen with SSRIs such as FLX and CIT. (C) URI did not affect the weights of the rats. *P<0.05, **P<0.01, Fisher's HSD tests, 7-12 rats per group.

FIGS. 8A-8E are graphs showing the effects of dietary supplementation with omega-3 fatty acids (OMG) on behaviors in the FST. During the first exposure to forced swimming, OMG supplementation had no effect on latencies to become immobile (A) or behavior subtypes (B), regardless of the length of pre-exposure. During the re-test, however, OMG exposure-dependently increased latencies to become immobile (C). OMG also exposure-dependently decreased immobility and increased swimming without affecting climbing (D), a pattern of behaviors similar to that seen with SSRIs. (E) The OMG treatments did not affect the weights of the rats. *P<0.05, **P<0.01, Fisher's HSD tests, 7-12 rats per group.

FIG. 9A-9E are graphs showing the effects of a normally subtherapeutically effective dose of URI (71.7 mg/kg) in rats that received normally subtherapeutically effective dietary supplementation with OMG (3 or 10 days) on behaviors in the FST. As expected, OMG supplementation had no effect on latencies to become immobile (A) or behavior subtypes (B) during the first exposure to forced swimming. During the re-test, however, this low dosage of URI increased latencies to become immobile (C) in rats given 10 but not 3 days of OMG supplementation. This low dosage of URI also decreased immobility and increased both swimming and climbing (D) in rats given 10 but not 3 days of OMG supplementation. This pattern of behaviors is different from that seen with TCAs or SSRIs. (E) Combined treatment with URI and OMG did not affect the weights of the rats. *P<0.05, **P<0.01, Fisher's HSD tests, 7-12 rats per group.

FIGS. 10A and 10B are graphs showing the effects of treatments with antidepressant-like efficacy in the FST on locomotor activity in rats given one exposure to forced swimming. (A) None of the treatments affected behavior when distance traveled in an open field (Mean±SEM, in cm) rather than swimming was measured during re-testing. (B) The weights of the rats did not differ among these treatments. *P<0.05, **P<0.01, Fisher's HSD tests, 6-8 rats per group.

### DETAILED DESCRIPTION OF THE INVENTION

The invention described herein features compositions and methods for the treatment of substance abuse disorders, such as alcohol and opiate abuse or dependence, psychiatric disorders, such as mood disorders (e.g., unipolar depression, dysthymia, cyclothymia, and bipolar disorder), attention-deficit hyperactivity disorder (ADHD), anxiety disorders (e.g., panic disorder and generalized anxiety disorder), obsessive-compulsive disorder (OCD), post-traumatic stress disorder (PTSD), phobias, and psychotic disorders (e.g., schizophrenia and schizoaffective disorder), and their symptoms, and other disorders, such as cardiovascular disease, cancer, dysmenorrhea, infertility, preeclampsia, postpartum depression, menopausal discomfort, osteoporosis, thrombosis, inflammation, hyperlipidemia, hypertension, rheumatoid arthritis, hyperglyceridemia, and gestational diabetes. The invention also features methods for enhancing neurodevelopment and delaying premature birth.

For these indications, the invention features the use of cytidine-containing, cytosine-containing, uridine-containing, creatine-containing, adenosine-containing, or adenosine-elevating compounds or omega-3 fatty acids. A preferred cytidine-containing compound is CDP-choline (also referred to as citicoline or CDP choline [sodium salt]), a preferred adenosine-containing compound is S-adenosylmethionine (SAMe), and a preferred uridine-containing compound is triacetyl uridine.

The cytidine-containing, cytosine-containing, uridine-containing, creatine-containing, adenosine-containing, or adenosine-elevating compounds may be co-administered with other compounds that are precursors for the synthesis of brain phospholipids, e.g., fatty acids (such as omega-3 fatty acids), lipids, or lecithin.

When combinations of the therapeutic agents described herein, e.g., an omega-3 fatty acid and uridine, are employed, unexpected synergistic effects are observed. Such combinations enable the use of a subtherapeutically effective amount of one or more of the components of the combination to achieve a therapeutic effect.

### Mood Disorders

Alterations in brain phospholipid metabolism may be involved in the pathophysiology of mood disorders such as depression, bipolar disorder, dysthymia, and cyclothymia. Because phospholipid metabolism affects the fluidity of neural membranes, it can play a critical role in extracellular processes including surface receptor binding and membrane-protein interactions, as well as intracellular processes including signal transduction and mitochondrial function (Pacheco et al. Prog Neurobiol 50:255-273 1996; Shetty et al. J Neurochem 67: 1702-1710 1996; Exton Eur J Biochem 243:10-20 1997; Nomura et al. Life Sci 68:2885-2891 2001). Depression has been linked to abnormalities in both membrane synthesis and fluidity (Moore et al. American Journal of Psychiatry 154:116-118 1997; Sonawalla et al. Am J Psychiatry 156:1638-1640 1999; Detke et al. Archives of General Psychiatry 57:937-943 2000; Moore et al. Bipolar Disorder 3:207-216 2000; Steingard et al. Biol Psychiatry 48:1053-1061 2000). Treatments that affect the metabolism of phospholipids or their incorporation into neural membranes may therefore have efficacy in the treatment of depression and other mood disorders.

Although there is evidence that treatments that affect phospholipid metabolism and membrane fluidity have some efficacy in the treatment of depressive symptoms, the effects are often modest and causal relationships are difficult to prove. For example, populations with diets rich in fish show lower prevalences of major depression (Hibbeln Lancet 351:1213, 1998). Fish is particularly high in omega-3 fatty acids, which are long-chain polyunsaturated fatty acids that are incorporated into neuronal membranes (for review, see Freeman Ann Clin Psychiatry 12:159-165, 2000). The double bonds within polyunsaturated fatty acids such as omega-3 fatty acids result in structural conformations that prevent dense packing of phospholipids, thereby influencing membrane fluidity (Popp-Snijders et al. Scand J Clin Lab Invest. 44: 39-46, 1984; Cartwright et al. Atherosclerosis 55:267-281, 1985). Treatment with omega-3 fatty acids in humans decreases brain water proton transverse relaxation times (T2s), consistent with increased membrane fluidity. Although omega-3 fatty acids have not been evaluated in controlled clinical trials of major depression, they improve the course of illness in patients with bipolar disorder, which involves depressive states (Stoll et al. Arch Gen Psychiatry 56: 407-412, 1999). Similarly, some symptoms of cocaine withdrawal, which often involves depressive symptoms, can be treated in clinical populations with citicoline (Renshaw et al. Psychopharmacology 142:132-138, 1999). Citicoline is metabolized in part to the nucleoside cytidine, which induces the biosynthetic pathways of structural membrane phospholipids and increases membrane production (Lopez-Coviella et al. J Neurochem 65:889-894, 1995; Knapp et al. Brain Res 822:52-59, 1999). Short-term administration of cytidine by systemic injection has antidepressant-like effects in rats (Carlezon et al. Biol. Psychiatry 51:882-889, 2002). Cytidine is further converted to the nucleoside uridine (Wurtman et al. Biochem Pharmacol 60:989-992, 2000), but neither of these agents has been examined in clinical studies of patients with mood disorders.

We have now discovered that CDP-choline is efficacious in human trials and that cytidine-containing and cytosine-containing compounds can be used to treat depression. CDP-choline has been found to have two important new therapeutic properties. First, CDP-choline improves brain chemistry, e.g., increases phospholipid synthesis, in healthy adults. This effect is particularly apparent in older adults. Second, CDP-choline has antidepressant effects that are similar to those of fluoxetine, a widely-used drug for the treatment of depression.

Cytidine-containing and cytosine-containing compounds are particularly efficacious in treating the elderly, and these compounds are efficacious in treating depression in patients with a co-morbid neurological disease (e.g., post-stroke depression). In addition, these compounds may be administered in conjunction with, and thereby work synergistically with, phospholipids (e.g., lecithin) or compounds that are precursors for the synthesis of brain phospholipids (e.g., fatty acids or lipids).

We have now also discovered that uridine and omega-3 fatty acids are efficacious, alone and in combination, in a treatment for unipolar depression or dysthymia. The therapeutic properties of uridine-containing compounds are similar to those of cytidine-containing compounds, while omega-3 fatty acids appear to produce an increase in membrane fluidity. In addition, the combination of a uridine-containing compound and an omega-3 fatty acid produces a synergistic effect, i.e., the combination of the two agents requires a reduced dose of each constituent.

### Substance Abuse or Dependence

Phosphorus-31 magnetic resonance spectroscopy (MRS) studies indicate that persons who are dependent upon alcohol and opiates have decreased brain levels of phospholipids. In addition, data derived from healthy older persons, indicates that chronic administration of CDP-choline is associated with neurochemical changes consistent with phospholipid synthesis. Increasing brain levels of cytosolic adenosine also provides effective therapy for alcohol or opiate abuse or dependency, because energy in the form of ATP is required to support phospholipid synthesis. Based on our results described herein, omega-3 fatty acids are utilized in another method of the invention to treat substance abuse or dependence, e.g., from alcohol, opiates, cocaine, amphetamines, methamphetamine, and methylphenidate. Omega-3 fatty acids may also be used in combination with other compounds as described herein.

### Attention Deficit Hyperactivity Disorder (ADHD)

Functional magnetic resonance imaging (fMRI) experiments in children diagnosed with ADHD indicate that symptoms of hyperactivity and inattention are strongly correlated with measures of blood flow within the putamen nuclei, which are strongly dopaminergic brain regions. In addition, administration of methylphenidate, a stimulant used to treat ADHD, increases blood flow in the putamen in parallel with a decrease in motor activity. ADHD symptoms may be closely tied to functional abnormalities in the putamen, which is predominantly involved in the regulation of motor behavior. Accordingly, because cytidine-containing and cytosine-containing compounds (e.g., CDP-choline) have dopaminergic activity, these compounds may be used to treat persons diagnosed with ADHD without many of the side effects associated with stimulant therapies. In particular, treatments with cytidine-containing or cytosine-containing compounds are effective in treating hyperactivity in children diagnosed with ADHD. Based on our results described herein, ADHD may also be treated with uridine-containing compounds, or a combination including an omega-3 fatty acid and either a cytidine-containing, cytosine-containing, uridine-containing, creatine-containing, adenosine-containing, or adenosine-elevating compound (e.g., a uridine-containing compound or a cytidine-containing compound), or a combination thereof.

### Other Psychiatric Disorders

Omega-3 fatty acids may be used in the treatment of other psychiatric disorders, such as anxiety disorders (e.g., panic disorder and generalized anxiety disorder) obsessive-compulsive disorder (OCD), post-traumatic stress disorder (PTSD), phobias, and psychotic disorders (e.g., schizophrenia and schizoaffective disorder). In these treatments, omega-3 fatty acids may be used in combination with a cytidine-containing, cytosine-containing, uridine-containing, creatine-containing, adenosine-containing, or adenosine-elevating compound.

### Neurodevelopment

The compounds of the invention may also be employed to enhance neurodevelopment, e.g., neurite growth. Exemplary combinations for this indication include an omega-3 fatty acid and a cytidine-containing, cytosine-containing, uridine-containing, creatine-containing, adenosine-containing, or adenosine-elevating compound. Methods for evaluating the enhancement of neurodevelopment are known in the art (e.g., Gibson, R. A. and M. Makrides Acta Paediatr, 1998, 87:1017-22, Fewtrell, M. S., et al., J Pediatr, 2004, 144:471-9, Fewtrell, M. S., et al., Pediatrics, 2002, 110:73-82, O′Connor, D. L., et al., Pediatrics, 2001, 108:359-71, Clandinin, M., et al., Pediatric Res, 2002, 51:187 A-8A, Innis, S. M., et al., J Pediatr, 2002, 140:547-54, Clandinin, M. T., et al., Pediatr Res, 1997, 42:819-25, Uauy, R., et al., J Pediatr, 1994, 124:612-20, Werkman, S. H. and S. E. Carlson, Lipids, 1996, 31:91-7., Carlson, S. E., et al., Eur J Clin Nutr, 1994, 48 Suppl 2:S27-30., Vanderhoof, J., et al, J Pediatr Gastroenterol Nutr, 2000, 31:121-7, and Marszalek, J. R., et al., J Biol Chem, 2004, 279:23882-91). Exemplary methods for gauging neurodevelopment include the Bayley Mental Developmental Index (MDI), the Bayley Psychomotor Developmental Index (PDI), Knobloch, Passamanick and Sherrard's Developmental Screening Inventory, and the Fagan Test of Infant Intelligence. Enhancement can be measured, for example, relative to a control group, such as a group that did not receive the compounds of the invention.

### Cardiovascular Disease

The compounds of the invention may also be employed to treat cardiovascular disease (CVD), including atherosclerosis, coronary artery disease, regression and decreased progression of coronary lesions, decrease in triglyceride blood levels, increase in HDL cholesterol, neutralization of LDL cholesterol, reduction in mortality from cardiac events, and decrease in ventricular tachycardia. Exemplary combinations for these indications include an omega-3 fatty acid and a cytidine-containing, cytosine-containing, uridine-containing, creatine-containing, adenosine-containing, or adenosine-elevating compound.

### Oncology

The compounds of the invention may also be employed to treat cancer, including reducing the risk of developing cancer (Larsson, S. C., et al., Am J Clin Nutr, 2004, 79:935-45), treating cancer cachexia during radio and chemotherapy and increasing the rate of recovery (Heller, A. R., et al., Int J Cancer, 2004, 111:611-6), and treating cancer-associated wasting (Jatoi, A., et al., J Clin Oncol, 2004, 22:2469-76). Exemplary cancers include breast, colon, pancreatic, chronic myelogenous leukemic, and melanoma. Exemplary combinations for these indications include an omega-3 fatty acid and a cytidine-containing, cytosine-containing, uridine-containing, creatine-containing, adenosine-containing, or adenosine-elevating compound.

### Women's Health

The methods of the invention also address a number of medical problems that exclusively or particularly effect women, e.g., dysmenorrhea, infertility (e.g., by increasing uterine blood flow), preeclampsia, postpartum depression, menopausal discomfort, and osteoporosis. The compounds of the invention may also be employed to delay premature birth, e.g., by balancing eicosanoids involved in labor and improving placental blood flow. Exemplary combinations for these indications include an omega-3 fatty acid and a cytidine-containing, cytosine-containing, uridine-containing, creatine-containing, adenosine-containing, or adenosine-elevating compound.

### Other Indications

The compounds of the inventions may also be used treat other indications, such as thrombosis, inflammation, hyperlipidemia, hypertension, rheumatoid arthritis, hyperglyceridemia, and gestational diabetes. Exemplary combinations for these indications include an omega-3 fatty acid and cytidine-containing, cytosine-containing, uridine-containing, creatine-containing, adenosine-containing, or adenosine-elevating compounds.

### Cytidine-Containing and Cytosine-Containing Compounds

Useful cytidine-containing or cytosine-containing compounds may include any compound including one of the following: cytosine, cytidine, CMP, CDP, CTP, dCMP, dCDP, and dCTP. Preferred cytidine-containing compounds include CDP-choline and cytidine 5′-diphosphocholine [sodium salt]. This list of cytidine-containing and cytosine-containing compounds is provided to illustrate, rather than to limit the invention, and the compounds described above are commercially available, for example, from Sigma Chemical Company (St. Louis, Mo.).

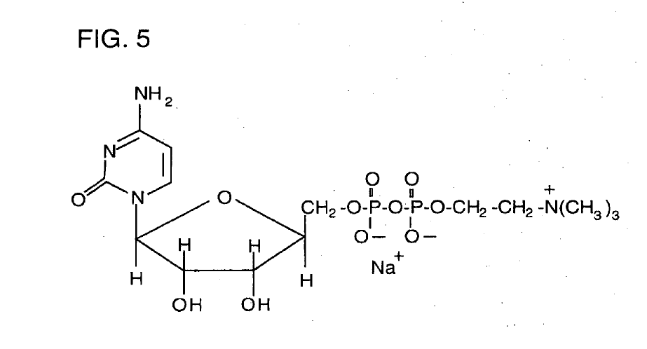

CDP-choline is a naturally occurring compound that is hydrolyzed into its components of cytidine and choline in vivo. CDP-choline is synthesized from cytidine-5′-triphosphate and phosphocholine with accompanying production of inorganic pyrophosphate in a reversible reaction catalyzed by the enzyme CTP:phosphocholine cytidylyltransferase (Weiss, Life Sciences 56:637-660, 1995). CDP-choline is available for oral administration in a 500 mg oblong tablet. Each tablet contains 522.5 mg CDP-choline sodium, equivalent to 500 mg of CDP-choline. Matching placebo tablets are also available. The excipients contained in both active and placebo tablets are talc, magnesium stearate, colloidal silicon dioxide, hydrogenated castor oil, sodium carboxy-methylcellulose, and microcrystalline cellulose. The molecular structure of CDP-choline [sodium salt] is provided in FIG. 5.

Other formulations for treatment or prevention of psychiatric and substance abuse disorders may take the form of a cytosine-containing or cytidine-containing compound combined with a pharmaceutically-acceptable diluent, carrier, stabilizer, or excipient.

### Adenosine-Containing and Adenosine-Elevating Compounds

Adenosine-containing or adenosine-elevating compounds provide useful therapies because these compounds provide the ATP needed for phospholipid synthesis. Useful adenosine-containing or adenosine-elevating compounds include, without limitation, any compound comprising one of the following adenosine, ATP, ADP, or AMP. One preferred adenosine-containing compound is S-adenosylmethionine (SAMe).

In addition, compounds are known that are capable of increasing adenosine levels by other mechanisms. For example, adenosine uptake can be inhibited by a number of known compounds, including propentofylline (described in U.S. Pat. No. 5,919,789, hereby incorporated by reference). Another known compound that inhibits adenosine uptake is EHNA.

Other useful compounds that can be used to increase brain adenosine levels are those that inhibit enzymes that break down adenosine, (e.g., adenosine deaminase and adenosine kinase). Finally, administering compounds that contain adenosine or precursors of adenosine, which are released as adenosine in vivo, can also be used.

### Uridine-Containing Compounds

Uridine and uridine-containing compounds may provide useful therapies because these compounds can be converted to CTP, a rate-limiting factor in PC biosynthesis (Wurtman et al., Biochemical Pharmacology 60:989-992, 2000). Useful uridine-containing compounds include, without limitation, any compound comprising uridine, UTP, UDP, or UMP. Uridine and uridine-containing compounds and analogs are well tolerated in humans. The oral bioavailability of uridine in humans can be increased by various means, e.g., acetylation of ring hydroxyl groups as in triacetyl uridine. Alternatively, formulations may be used to increase bioavailbility.

### Creatine-Containing Compounds

Creatine and creatine-containing compounds provide useful therapies because these compounds, by virtue of increasing brain phospholipid levels, can raise the levels of ATP. Creatine and creatine-containing compounds are known to be well tolerated at relatively high doses in humans.

### Omega-3 Fatty Acids

Omega-3 fatty acids provide useful therapy likely because they increase membrane fluidity. Exemplary omega-3 fatty acids include eicosapentaenoic acid, docosahexaenoic acid, and α-linolenic acid. Omega-3 fatty acids may be administered as the free acid, a salt, or in esterified form (e.g., as triglycerides or phospholipids). Omega-3 fatty acids may be obtained in pure form by synthesis or by culture of microalgae. Omega-3 fatty acids may also be administered in a mixture from a naturally occurring source, e.g., fish oil, flaxseed oil, soybeans, rapeseed oil, or microalgae. The use of omega-3 fatty acids with other therapeutic compounds of the invention may produce a synergistic effect, i.e., the combination of the two agents requires a reduced dose of each constituent.

### Administration

Conventional pharmaceutical practice is employed to provide suitable formulations or compositions for administration to patients. Oral administration is preferred, but any other appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, or aerosol administration. Therapeutic formulations may be in the form of liquid solutions or suspensions (as, for example, for intravenous administration); for oral administration, formulations may be in the form of liquids, tablets, or capsules; and for intranasal formulations, in the form of powders, nasal drops, or aerosols. In particular, omega-3 fatty acids may be administered in an inclusion complex, dispersion (such as a micelle, microemulsion, and emulsion), or liposome, for example, as described in U.S. Application No. 10/______, titled “ENHANCED EFFICACY OF OMEGA-3 FATTY ACID THERAPY IN THE TREATMENT OF PSYCHIATRIC DISORDERS,” filed on Oct. 8, 2004. In addition, compounds useful in the methods described herein also include encapsulated compounds, e.g., liposome- or polymer-encapsulated cytidine-containing, cytosine-containing, uridine-containing, creatine-containing, adenosine-containing, and adenosine-elevating compounds. Useful compounds further include those linked (e.g., covalently or non-covalently) to various antibodies, ligands, or other targeting and enveloping or shielding agents (e.g., albumin or dextrose), to allow the cytidine-containing, cytosine-containing, uridine-containing, creatine-containing, adenosine-containing, or adenosine-elevating compound to reach the target site (e.g., the central nervous system) prior to being removed from the blood stream, e.g., by the kidneys and liver, and prior to being degraded.

Methods well known in the art for making formulations are described, for example, in Remington: The Science and Practice of Pharmacy (20th ed.) ed. A.R. Gennaro, Lippincott: Philadelphia 2003. Formulations for parenteral administration may, for example, contain excipients, sterile water, saline, polyalkylene glycols such as polyethylene glycol, oils of vegetable origin, or hydrogenated naphthalenes.

If desired, slow release or extended release delivery systems may be utilized. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Preferably, the compounds of the invention, such as CDP-choline, are administered at a dosage of at least 500 mg twice daily by oral administration. Orally administered CDP-choline is bioavailable, with more than 99% of CDP-choline and/or its metabolites absorbed and less than 1% excreted in feces. CDP-choline, administered either orally or intravenously, is rapidly converted into the two major circulating metabolites, choline and cytidine. Major excretion routes are lung (12.9%) and urine (2.4%); the rest of the dose (83.9%) is apparently metabolized and retained in tissues.

In general, the compounds of the invention, such as CDP-choline, uridine, UTP, creatine, or SAMe, are administered at a dosage appropriate to the effect to be achieved and are typically administered in unit dosage form. The dosage preferably ranges from 50 mg per day to 2000 mg per day. The exact dosage of the compound may be dependent, for example, upon the age and weight of the recipient, the route of administration, and the severity and nature of the symptoms to be treated. In general, the dosage selected should be sufficient to prevent, ameliorate, or treat a particular indication, or one or more symptoms thereof, or effect a particular outcome without producing significant toxic or undesirable side effects. As noted above, the preferred route of administration for most indications is oral.

In the case of CDP-choline, there have been no reported cases of overdoses. CDP-choline toxicity is largely self-limiting, ingestion of large amounts in preclinical studies shows common cholinergic symptoms (salivation, lacrimation, urination, defecation, and vomiting).

Combination with Other Therapeutics

The cytidine-containing, cytosine-containing, uridine-containing, creatine-containing, adenosine-containing, adenosine-elevating compounds, and omega-3 fatty acids of the invention may be administered as a monotherapy, in combination with each other, or in combination with other medicaments for the indications described herein.

Preferably, the compounds of the invention may be administered in conjunction with lower doses of current medicaments for these indications, including stimulants and antidepressants. For example, the compounds of the invention may be administered with phospholipids, e.g., lecithin, or with brain phospholipid precursors, e.g., fatty acids or lipids, or may be administered as an adjunct to standard therapy for the treatment of psychiatric or substance abuse disorders.

In one particular example, the compound of the invention may be administered in combination with an antidepressant, anticonvulsant, antianxiety, antimanic, antipyschotic, antiobsessional, sedative-hypnotic, stimulant, or anti-hypertensive medication. Examples of these medications include, but are not limited to, the antianxiety medications, alprazolam, buspirone hydrochloride, chlordiazepoxide, chlordiazepoxide hydrochloride, clorazepate dipotassium, desipramine hydrochloride, diazepam, halazepam, hydroxyzine hydrochloride, hydroxyzine pamoate, lorazepam, meprobamate, oxazepam, prazepam, prochlorperazine maleate, prochlorperazine, prochlorperazine edisylate, and trimipramine maleate; the anticonvulsants, amobarbital, amobarbital sodium, carbamazepine, chlordiazepoxide, chlordiazepoxide hydrochloride, clorazepate dipotassium, diazepam, divalproex sodium, ethosuximide, ethotoin, gabapentin, lamotrigine, magnesium sulfate, mephenyloin, mephobarbital, methsuximide, paramethadione, pentobarbital sodium, phenacemide, phenobarbital, phenobarbital sodium, phensuximide, phenyloin, phenyloin sodium, primidone, secobarbital sodium, trimethadione, valproic acid, and clonazepam; the antidepressants, amitriptyline hydrochloride, amoxapine, bupropion hydrochloride, clomipramine hydrochloride, desipramine hydrochloride, doxepin hydrochloride, fluoxetine, fluvoxamine, imipramine hydrochloride, imipramine pamoate, isocarboxazid, lamotrigine, maprotoline hydrochloride, nortriptyline hydrochloride, paroxetine hydrochloride, phenelzine sulfate, protriptyline hydrochloride, sertraline hydrochloride, tranylcypromine sulfate, trazodone hydrochloride, trimipramine maleate, and venlafaxine hydrochloride; the antimanic medications, lithium carbonate and lithium citrate; the antiobsessional medications, fluvoxamine, and clomipramine hydrochloride; the antipsychotic medications, acetophenazine maleate, chlorpromazine hydrochloride, chlorprothixene, chlorprothixene hydrochloride, clozapine, fluphenazine decanoate, fluphenazine enathrate, fluphenazine hydrochloride, haloperidol decanoate, haloperidol, haloperidol lactate, lithium carbonate, lithium citrate, loxapine hydrochloride, loxapine succinate, mesoridazine besylate, molindone hydrochloride, perphenazine, pimozide, prochlorperazine maleate, prochlorperazine, prochlorperazine edisylate, promazine hydrochloride, risperidone, thioridazine, thioridazine hydrochloride, thiothixene, thiothixene hydrochloride, and trifluoperzine hydrochloride; the sedative-hypnotic medications, amobarbital, amobarbital sodium, aprobarbital, butabarbital, chloral hydrate, chlordiazepoxide, chlordiazepoxide hydrochloride, clorazepate dipotassium, diazepam, diphenhydramine, estazolam, ethchlorvynol, flurazepam hydrochloride, glutethimide, hydroxyzine hydrochloride, hydroxyzine pamoate, lorazepam, methotrimeprazine hydrochloride, midazolam hydrochloride, oxazepam, pentobarbital sodium, phenobarbital, phenobarbital sodium, quazepam, secobarbital sodium, temazepam, triazolam, and zolpidem tartrate; the stimulants, dextroamphetamine sulfate, methamphetamine hydrochloride, methylphenidate hydrochloride, and pemoline; and the anti-hypertensive, clonidine.

The following examples are provided for the purpose of illustrating the invention and should not be construed as limiting.

### Unipolar Depression or Dysthymia

Treatment of Human Subjects with Cytidine- or Cytosine-Containing Compounds

Proton and phosphorus magnetic resonance (MR) spectroscopy studies of subjects with mood disorders have characterized two patterns of altered neurochemistry associated with depression. The first pattern indicates a change (increase or decrease) in cytosolic choline, as well as increased frontal lobe phosphomonoesters, while the second pattern points to decreased brain purines (cytosolic adenosine-containing compounds) and decreased nucleoside triphosphates (NTP). The former results reflect altered phospholipid metabolism, while the latter results indicate changes in cerebral energetics. Although few longitudinal studies have been conducted, these altered metabolite levels appear to be mood state, rather than trait, dependent.

To assess whether chronic CDP-choline administration leads to detectable changes in lipid metabolite resonances in phosphorus-31 MR spectra, eighteen healthy subjects (mean age: 70) were administered 500 mg of an oral formulation of CDP-choline daily for a six week period. From weeks 6 to 12, half of the subjects continued to receive CDP-choline and half received placebo in a double-blind fashion. The MR data demonstrated that CDP-choline treatment was associated with a significant increase in brain phosphodiesters (p=0.008), a finding that is indicative of increased phospholipid synthesis. Neuropsychological testing also revealed increases in verbal fluency (p=0.07), verbal learning (p=0.003), visuospatial learning (p=0.0001) across all subjects at week twelve. CDP-choline administration, therefore, improves measures of verbal fluency and spatial memory in healthy adults and results in increased brain phospholipid synthesis in older adults, particularly during chronic administration.

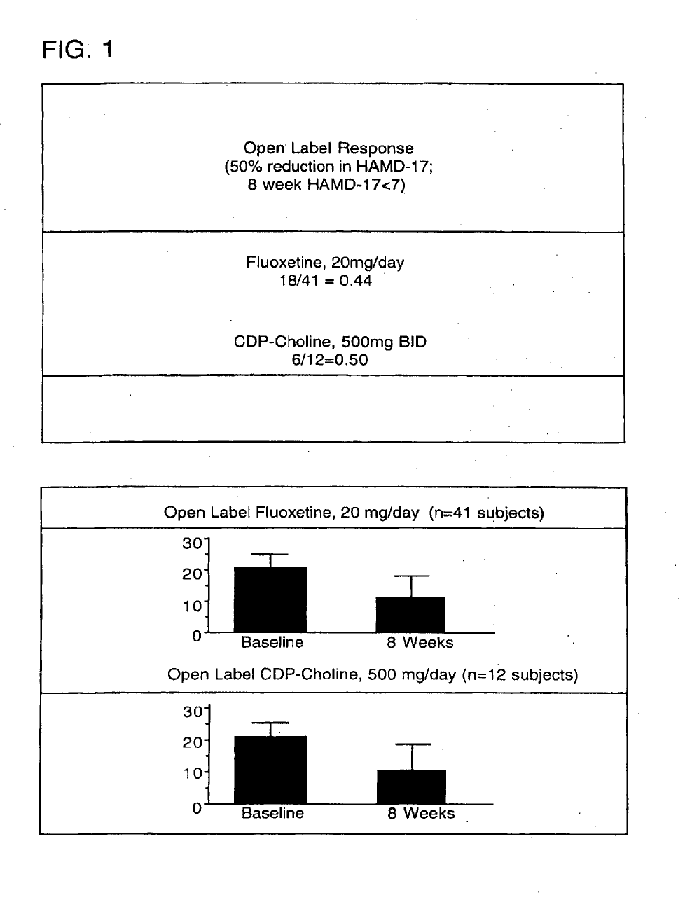

In a second study, twelve depressed subjects (mean age 40) received 500 mg of an oral formulation of CDP-choline twice daily for eight weeks. With eight weeks of treatment, mean 17-item Hamilton Depression Rating Scale (HDRS) scores decreased from 21±3 to 10±7 (p<0.0001). A successful response to CDP-choline was also associated with a reduction in the proton MR spectroscopic cytosolic choline resonance in the anterior cingulate cortex. Comparable data for forty-one depressed subjects participating in imaging trials and treated with open label fluoxetine, 20 mg/day for eight weeks, demonstrated reductions in HDRS scores from 21±4 to 11±6 (p<0.0001) (FIG. 1). CDP-choline and fluoxetine were associated with complete responses in 6/12 (50%) and 17/41 (41%) of the subjects, respectively (FIG. 1). In depressed adults, therefore, the antidepressant effects of CDP-choline were comparable to those of fluoxetine.

These data represent the first demonstration that human brain lipid metabolism can be modified using pharmacological strategies, and that, particularly in older adults, treatment is associated with improved cognitive performance. These data demonstrate that therapeutic strategies, using cytosine- and cytidine-containing compounds (e.g., CDP-choline), that are aimed at reversing biochemical alterations are beneficial for the treatment of depression or dysthymia.

### Use of Citicoline in a Rodent Model of Depression

The effects of citicoline were examined in the forced swim test (FST), a rodent model of depression as described herein. Because citicoline is rapidly converted to cytidine and choline, their effects were also examined in the FST. Citicoline did not have antidepressant effects in rats in the FST over a range of doses (50-500 mg/kg, IP) shown to have neuroprotective effects in experimental ischemia in rodents. In fact, high doses of citicoline appeared to have small pro-depressant effects in this model. Molar equivalent amounts of cytidine (23.8-238 mg/kg, IP) had significant antidepressant effects in the FST, whereas molar equivalent amounts of choline (13.7-136.6 mg/kg, IP) had significant pro-depressant effects. The optimally effective dose of cytidine (238 mg/kg, IP) did not affect locomotor activity or establish conditioned rewarding effects at therapeutic concentrations.

### Use of Uridine and Omega-3 Fatty Acids in a Rat Model of Depression

The behavioral effects of the combination of uridine and omega-3 fatty acids were also evaluated in rats using the forced swim test (FST). This assay identifies in rodents treatments that have antidepressant effects in humans (Porsolt et al. Nature 266:730-732 1977; Carlezon et al. Biol. Psychiatry 51:882-889, 2002). Uridine was administered using systemic injection while omega-3 fatty acids were administered by supplementation within the diet for various periods of time (3, 10, or 30 days). The effects of uridine in rats maintained on the omega-3 fatty acid-enriched diet were also evaluated to determine if these effects were additive. For comparison, the effects of the standard antidepressant drugs desipramine (a tricyclic antidepressant [TCA]) and fluoxetine and citalopram (selective serotonin reuptake inhibitors [SSRIs]) were determined. The efficacy of each treatment in the FST was evaluated using two separate scoring methods: latency to become immobile, a simple and rapid method that identifies agents with antidepressant effects (Pliakas et al. J Neurosci 21:7397-7403 2001), and behavioral sampling, a more complex method that differentiates antidepressant drugs according to their pharmacological mechanisms (Detke et al. Psychopharmacology 121:66-72 1995). Finally, treatments with antidepressant-like effects in the FST were evaluated for non-specific effects on activity in an open field, which might complicate interpretation of the data from the swimming studies.

### Methods

Rats: A total of 197 male Sprague-Dawley rats (Charles River Laboratories, Boston Mass.) were used in these studies. The rats were housed in groups of four and weighed 325-375 gm at the time of behavioral testing. Rats were maintained on a 12 h light (0700-1900 h)-12 h dark cycle with free access to food and water except during testing. Experiments were conducted in accordance with the 1996 Guide for the Care and Use of Laboratory Animals (NIH) and McLean Hospital policies.

Drugs: Dosages of desipramine HCl (DMI), fluoxetine HCl (FLX), citalopram HBr (CIT), and uridine (URI) were administered in a distilled water vehicle (VEH) at a volume of 1 cc/kg. All drugs were purchased from RBI-Sigma (St. Louis, Mo.) except CIT, which was a gift of Forest Laboratories (New York, N.Y.). Fatty acids were administered as a dietary supplement in food fortified with either menhaden oil (OMG) containing omega-3 fatty acids, or olive oil (CON), as a control, each at 4.5% w/w (Research Diets Inc., New Brunswick N.J.). The menhaden oil contained 27% w/w omega-3 fatty acids, and the rats ate an average of 25 gm of food (0.3 gm OMG) each day. The diets were equivalent in overall fat, protein, carbohydrate, and caloric content.

Forced Swim Test (FST): One hundred-sixty seven rats were used in the FST studies, which were conducted as described previously (Carlezon et al. Biol. Psychiatry 51:882-889, 2002) with minor modifications. The FST is a two-day procedure in which rats swim under conditions in which escape is not possible. On the first day, rats are placed in clear, 65 cm tall-25 cm diameter cylinders filled to 48 cm with 25° C. water. The rats initially struggle to escape from the water, but eventually they adopt a posture of immobility in which they make only the movements necessary to keep their heads above water. After 15 min of forced swimming, the rats are removed from the water, dried with towels, and placed in a warmed enclosure for 30 min. The cylinders are emptied and cleaned between rats. When the rats are re-tested 24 hours later under identical conditions in 5 min sessions, immobility is increased. Treatment with standard antidepressant drugs within the 24 hr period between the first exposure to forced swimming and re-testing can attenuate facilitated immobility, an effect correlated with antidepressant efficacy in humans (Porsolt et al. Nature 266:730-732 1977; Detke et al. Psychopharmacology 121:66-72 1995, Carlezon et al. Biol. Psychiatry 51:882-889, 2002).

Rats tested with DMI, FLX, CIT, or URI received 3 separate intraperitoneal (IP) injections of drug (or VEH), at 1 hr, 19 hr, and 23 hr after the first exposure to forced swimming. This commonly used regimen is sensitive to the antidepressant-like effects of many standard agents (Porsolt et al. Nature 266:730-732 1977; Detke et al. Psychopharmacology 121:66-72 1995; Carlezon et al. Biol. Psychiatry 51:882-889, 2002). Rats tested with OMG (or CON) received the special diets 3, 10, or 30 days prior to the start of the swim test, and received saline or URI injections (IP) at 1, 19, and 23 hr after the forced swim. There were 7-12 rats per treatment condition, and separate rats were used for each treatment regimen.

Swim tests were videotaped from the side of the cylinders, and scored by raters unaware of the treatment conditions. The re-test (day 2) of the FST was videotaped for the groups receiving only DMI, FLX, CIT, URI, or VEH injections because these rats had not received any treatments before the first exposure to forced swimming. Both days of FST testing were videotaped for rats that were maintained on the special diets because the groups differed before the first exposure to forced swimming. Rats were scored using two separate but complementary methods: latency to immobility and behavioral sampling. Latency to become immobile was defined as the time at which the rat first initiated a stationary posture that did not reflect attempts to escape from the water. In this characteristic posture, the forelimbs are motionless and tucked toward the body. To qualify as immobility, this posture had to be clearly visible and maintained for 2.0 sec. For behavioral sampling, rats were rated at 5 sec intervals throughout the duration of the forced swimming session. At each 5 sec interval, the predominant behavior was assigned to one of 4 categories: immobility, swimming, climbing, or diving (Detke et al. Psychopharmacology 121:66-72 1995). A rat was judged to be immobile if it was making only movements necessary to keep its head above water, climbing if it was making forceful thrashing movements with its forelimbs directed against the walls of the cylinder, swimming if it was actively making swimming movements that caused it to move within the center of the cylinder, and diving if it swam below the water, toward the bottom of the cylinder. Diving behavior rarely occurred, and it was not affected by any of the treatments tested. The behavioral sampling method reportedly differentiates classes of antidepressant drugs: for example, TCAs decrease immobility and increase climbing without affecting swimming, whereas SSRIs decrease immobility and increase swimming without affecting climbing (Detke et al. Psychopharmacology 121:66-72 1995).

Data from the tests with the standard agents (DMI, FLX and CIT) were analyzed together, whereas data from the tests with URI alone were analyzed separately. For these treatments, latencies to become immobile or the number of occurrences of each category of behavior was analyzed using separate one-way (treatment) analyses of variance (ANOVAs). Significant effects were analyzed further using post hoc Fisher's honestly significant difference (HSD) tests. Data from the tests with OMG alone and OMG plus URI were analyzed separately, and each day of testing was analyzed independently. For these treatment regimens, latencies to become immobile or the number of occurrences of each category of behavior was analyzed using separate two-way (treatment×duration of diet) analyses of variance (ANOVAs), followed by post hoc Fisher's HSD tests.

Locomotor activity: Thirty rats were used to determine if the treatments that were effective in the FST studies had non-specific effects on activity levels in rats exposed previously to forced swimming. These studies were conducted exactly as the FST studies had been conducted until the time of re-testing: that is, all rats underwent the first day of the FST, but 24 hr later they were placed for 1 hr in automated, 17×17×12 in (L×W×H) open field activity chambers (Med Associates, St. Albans Vt.) instead of being re-exposed to forced swimming. There were 6-8 rats per treatment condition; control rats received injections of VEH. The total distance traveled (in cm) during the test session was quantified, and data were analyzed with a one-way (treatment) ANOVA followed by post hoc Fisher's HSD tests. The researchers who established the FST interpreted the facilitated immobility during the second exposure to forced swimming as an indicator of “behavioral despair,” a depressive-like symptom (Porsolt et al. Nature 266:730-732, 1977). Regardless of the etiology of facilitated immobility, all of the major classes of antidepressant treatments—including TCAs, SSRIs, atypicals, monoamine oxidase inhibitors, and electroconvulsive shock therapy (Porsolt et al. Nature 266:730-732, 1977; Borsini et al. Psychopharmacol 94:147-160, 1988; Detke et al. Psychopharmacology 121:66-72, 1995)—effectively reduce indicators of immobility in the FST. Indeed, the main strength of the FST is its ability to identify, in rats, treatments with antidepressant efficacy in people (Willner Psychopharmacology 83:1-16, 1984). DMI, FLX and CIT reduced immobility when given by injection within the time between the first and second exposure to forced swimming. A similar treatment regimen with uridine also reduced indicators of immobility in the FST, indicating that this agent has antidepressant-like effects in rats. Rats fed a diet enriched with omega-3 fatty acids were also less immobile in the FST, consistent with antidepressant-like effects. A normally sub-effective dose of uridine had antidepressant-like effects in rats given a normally sub-effective treatment regimen of dietary supplementation with omega-3 fatty acids, suggesting that the antidepressant-like effects of these two treatments can potentiate one another. Considered together, these data provide strong evidence in an animal model that treatments that affect phospholipid metabolism and membrane fluidity may have promise in the treatment of depressive-like symptoms in humans.

### Results

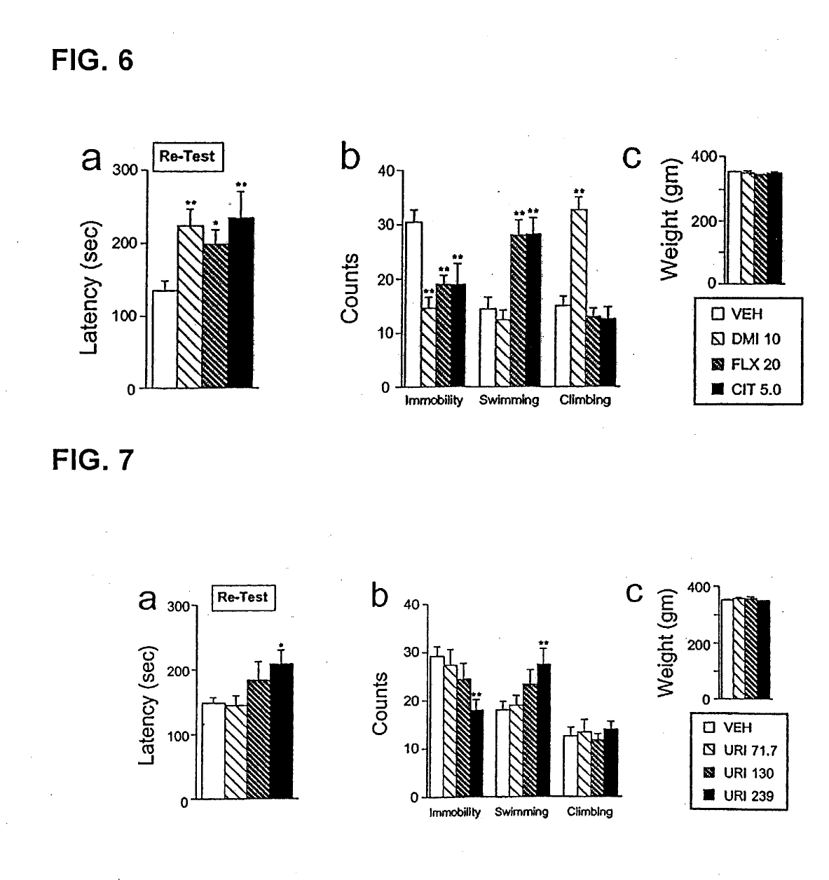

Standard antidepressant treatments (DMI, FLX, CIT) reduced indicators of immobility in the FST during the re-test (day 2), regardless of the scoring method that was used. These agents affected latencies to become immobile (F_{3,39}=5.73, P<0.01) when this method of scoring was used (FIG. 6A): the amount of time that elapsed before the first bout of immobility was increased by DMI (10 mg/kg; P<0.01, Fisher's HSD), FLX (20 mg/kg; P<0.05) and CIT (5.0 mg/kg; P<0.01). These agents also affected the patterns of behavior when the sampling method was used (FIG. 6B): they caused differences in the number of occurrences of immobility (F_{3,39}=9.14, P<0.01), swimming (F_{3,39}=10.3, P<0.01), and climbing (F_{3,39}=16.1, P<0.01) behaviors. Consistent with previous observations (Detke et al. Psychopharmacology 121:66-72, 1995), DMI (a TCA) reduced immobility and increased climbing (P′ s<0.01) without affecting swimming, whereas FLX and CIT (SSRIs) reduced immobility and increased swimming (P′ s<0.01) without affecting climbing. The weights of the rats did not differ among groups at the time of the re-test (FIG. 6C), which is important because weight can influence swimming behaviors (Pliakas et al. J Neurosci 21:7397-7403, 2001).

URI had dose-dependent effects on latencies to become immobile (F_{3,32}=3.05, P <0.05) (FIG. 7A): this agent increased latencies at 239 mg/kg (P<0.05), but not at 130 or 71.7 mg/kg. With the behavioral sampling method (FIG. 7B), URI significantly affected the occurrences of immobility (F_{3,32}=3.10, P<0.05) and swimming (F_{3,32}=3.07, P<0.05) without affecting climbing. URI reduced immobility (P<0.01) and increased swimming behaviors (P<0.01) at 239 mg/kg only. This pattern of behaviors is similar to that seen after treatment with SSRIs. The weights of the rats did not differ among groups at the time of the re-test (FIG. 7C).

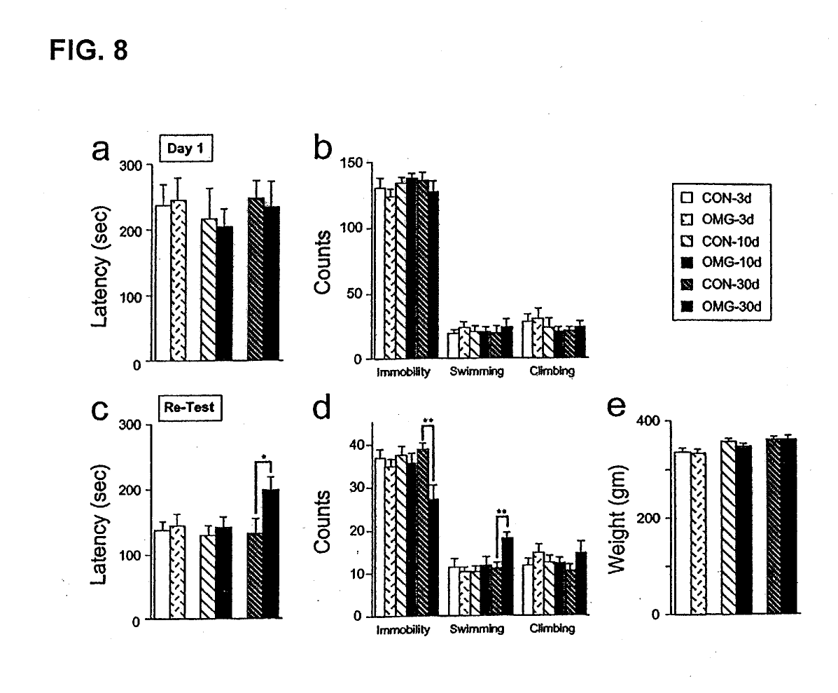

The effects of dietary supplementation with OMG alone depended upon the length of treatment, and were apparent only during the re-test session. During the first exposure to forced swimming, dietary OMG had no effect on latencies to become immobile (FIG. 8A) or any of the behavior subtypes (FIG. 8B). During the re-test, however, OMG affected latencies to become immobile (Main effect of treatment: F_{1,50}=4.08, P<0.05) (FIG. 8C): latencies were elevated in rats that had received OMG for 30 days (P<0.05), but not for 10 or 3 days. Similarly, OMG significantly affected occurrences of immobility (treatment×duration interaction: F_{1,50}=3.22, P<0.05) and swimming (treatment×duration interaction: F_{1,50}=3.42, P<0.05) without affecting climbing (FIG. 8D). OMG reduced immobility (P<0.01) and increased swimming behaviors (P<0.01) after 30 days treatment only. This pattern of behaviors is similar to that seen after treatment with SSRIs. The weights of the rats did not differ between treatment groups at the time of the re-test (FIG. 8E).

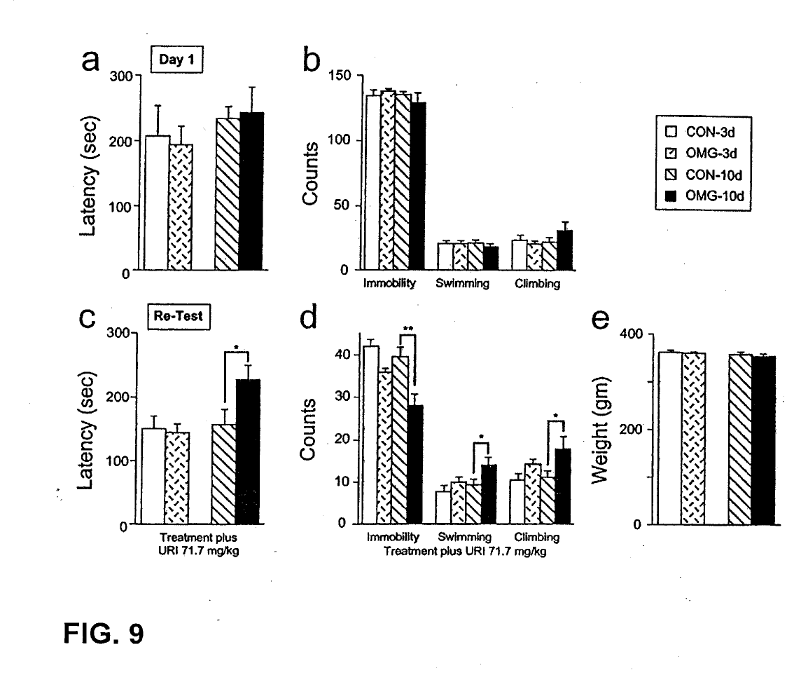

Administration of a sub-effective dosage of URI affected behavior in rats maintained on a normally sub-effective regimen of OMG dietary supplementation. Confirming earlier observations, OMG supplementation for 3 or 10 days had no effects on behaviors during the first exposure to forced swimming (FIG. 9A-9B). During the re-test, however, latencies to become immobile were altered in OMG-fed rats that also received 71.7 mg/kg URI (Main effect of duration: F_{1,28}=4.52, P<0.05) (FIG. 9C): latencies were elevated in rats that had received URI after OMG supplementation for 10 days (P<0.05), but not for 3 days. Likewise, the combination of normally sub-effective treatments with URI and OMG affected immobility (Main effect of treatment: F_{1,28}=17.7, P<0.01) , swimming (Main effect of treatment: F_{1,28}=6.46, P<0.02), and climbing (treatment×duration interaction: F_{1,28}=7.77, P<0.01) behaviors (FIG. 9D). URI treatment reduced immobility (P<0.01), increased swimming (P<0.05) and increased climbing (P<0.05) in rats given 10 days, but not 3 days, of OMG. The weights of the rats did not differ between treatment groups at the time of the re-test (FIG. 9E).

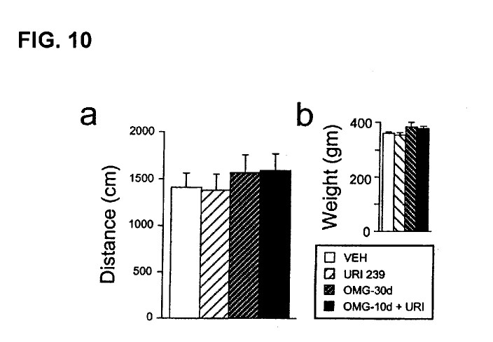

None of the treatments with antidepressant-like effects in the FST affected activity levels when rats were tested in open field chambers rather than the forced swim cylinders during the re-test (FIG. 10A). The weights of the rats did not differ among these groups (FIG. 10B).

The FST in rats is a useful model for predicting beneficial effects of therapies for depression in humans. The effects of uridine in the FST are similar to those for equimolar concentrations of cytidine. The mechanisms by which uridine and cytidine have antidepressant-like effects in the FST are unknown. One possibility is that these nucleosides affect the synthesis or fluidity of neural membranes (Lopez-Coviella et al. J Neurochem 65:889-894, 1995; Knapp et al., 1999; Wurtman et al. Biochem Pharmacol 60:989-992, 2000), each of which may be anomalous in mood disorders (Moore et al. American Journal of Psychiatry 154:116-118 1997; Sonawalla et al. Am J Psychiatry 156:1638-1640 1999; Detke et al. Archives of General Psychiatry 57:937-943 2000; Moore et al. Bipolar Disorder 3:207-216 2000; Steingard et al. Biol Psychiatry 48:1053-1061 2000). Another possibility is that the actions of uridine are mediated through its ability to alter catecholamine function in the brain. While the effects of uridine per se on catecholamine function are not known, citicoline increases brain production of neurotransmitters such as norepinephrine and dopamine, possibly by affecting precursors such as tyrosine (Martinet et al. Arch Int Pharmacodyn 239: 52-56 1979). To begin exploring the mechanisms by which uridine has antidepressant-like effects, we scored the FST using behavioral sampling, a detailed scoring method that can differentiate between various classes of antidepressant agents (Detke et al. Psychopharmacology 121:66-72 1995). Consistent with previous studies in which behavioral sampling was used (Detke et al. Psychopharmacology 121:66-72 1995), the standard norepinephrine uptake inhibitor desipramine decreased measures of immobility and increased measures of climbing without affecting measures of swimming. Conversely, the standard SSRIs fluoxetine, and citalopram decreased immobility and increased swimming without affecting climbing. Although differential effects on the swimming and climbing measures may involve factors other than norepinephine-serotonin interactions, the effects of uridine in the FST resemble those of fluoxetine and citalopram (altered immobility and swimming) rather than those of desipramine (altered immobility and climbing) indicating that uridine may be effective in this assay because of effects on serotonergic function.

The mechanisms by which omega-3 fatty acids have antidepressant-like effects are unknown. Omega-3 fatty acids appear to have profound effects on the fluidity of neural membranes. Importantly, the antidepressant-like effects of omega-3 fatty acids were seen only with long-term dietary enrichment, and not after shorter regimens. These results may explain the subtle effects of omega-3 fatty acids in humans, and highlight the challenges that complicate clinical studies with this type of agent. Furthermore, the effects were not seen in the rats during the first exposure to forced swimming, but only during the re-test. Inasmuch as facilitated immobility in the FST is due to activation of intracellular signaling pathways and genes associated with stress (Pliakas et al. J Neurosci 21:7397-7403, 2001), these findings suggest that omega-3 fatty acids interfere with the induction of neuroadaptations that contribute to development of immobility behaviors that may reflect learned helplessness.

Treatment with low dosages of uridine made shorter treatment regimens of omega-3 fatty acids effective in the FST. Although the mechanisms of this interaction are unknown, it seems likely that the effects of nucleosides on membrane synthesis (Lopez-Coviella et al. J Neurochem 65:889-894, 1995; Knapp et al., 1999; Wurtman et al. Biochem Pharmacol 60:989-992, 2000) may facilitate the incorporation of omega-3 fatty acids into neural membranes, where they can affect extracellular processes including surface receptor binding and membrane-protein interactions, as well as intracellular processes including signal transduction and mitochondrial function (Pacheco et al. Prog Neurobiol 50:255-273 1996; Shetty et al. J Neurochem 67: 1702-1710 1996; Exton Eur J Biochem 243:10-20 1997; Nomura et al. Life Sci 68:2885-2891 2001). The effects on membrane fluidity may be particularly important within mitochondria, which are vital for energy metabolism and have a high concentration of polyunsaturated fatty acids within their inner phospholipid membranes (Buttriss et al. Biochim Biophys Acta 962:81-90, 1988; Raederstorff et al. Lipids 26:781-787, 1991). Indeed, dysregulation of mitochondria function is suspected in depression-related syndromes such as bipolar disorder (Kato et al. Bipolar Disorder 2:180-190, 2000), and individuals with bipolar disorder appear to benefit from omega-3 fatty acid therapy (Stoll et al. Arch Gen Psychiatry 56: 407-412, 1999).

### Alcohol or Opiate Abuse or Dependence

### Measurement of Brain Phospholipids

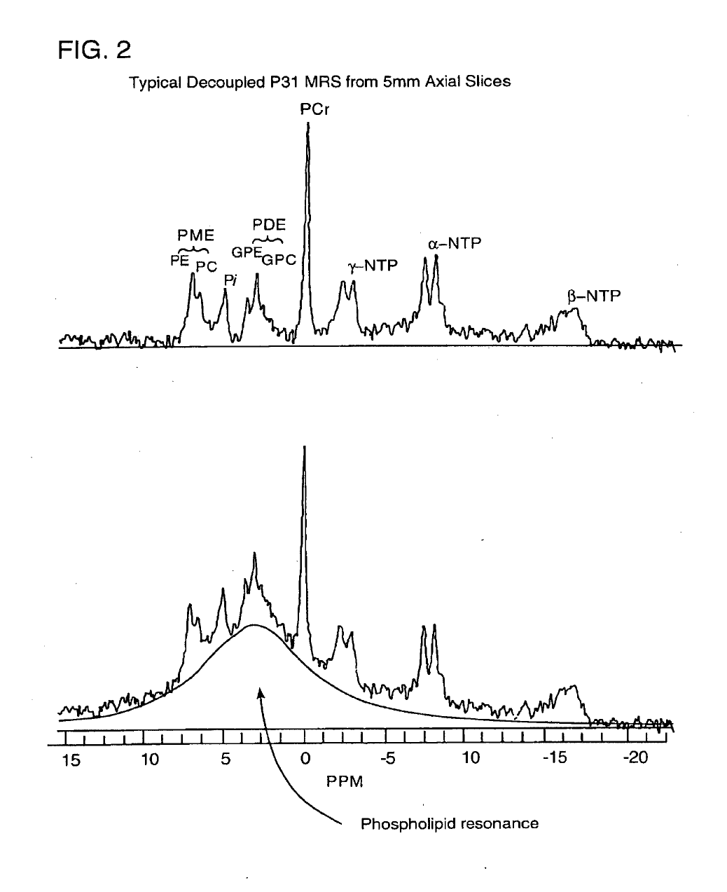

The broad component within the phosphorus-31 MR spectrum, arising from human brain phospholipids, may be measured reliably (FIG. 2). Preliminary results indicate that in persons with alcohol and/or opiate dependence, the intensity of this broad phospholipid resonance is decreased by 10-15% relative to values for comparison subjects. Accordingly, therapeutic strategies that are aimed at reversing this biochemical alteration, for example, by increasing phospholipid synthesis, are beneficial for the treatment of alcohol and/or opiate dependence.

### CDP-Choline Administration Leads To Increased Phospholipid Synthesis

To assess whether chronic CDP-choline administration leads to detectable changes in lipid metabolite resonances in phosphorus-31 MR spectra, eighteen healthy subjects (mean age: 70) were administered 500 mg of an oral formulation of CDP-choline daily for a six week period. From weeks 6 to 12, half of the subjects continued to receive CDP-choline and half received placebo in a double-blind fashion. The MR data demonstrated that CDP-choline treatment was associated with a significant increase in brain phosphodiesters (p=0.008), a finding that is indicative of increased phospholipid synthesis. Neuropsychological testing also revealed increases in verbal fluency (p=0.07), verbal learning (p=0.003), visuospatial learning (p=0.0001) across all subjects at week twelve. CDP-choline administration, therefore, improves measures of verbal fluency and spatial memory in healthy adults and results in increased brain phospholipid synthesis in older adults, particularly during chronic administration.

### Attention Deficit Hyperactivity Disorder (ADHD)

Functional Magnetic Resonance Imaging of Children Diagnosed with ADHD

A new fMRI procedure (T2 relaxometry or “T2-RT”) was developed to indirectly assess blood volume in the striatum (caudate and putamen) of boys 6-12 years of age under steady-state conditions. Six healthy control boys (10.2±1.5 yr) and eleven boys diagnosed with ADHD (9.3±1.6 yr) served as subjects in the study to examine fMRI differences between unmedicated healthy controls and ADHD children on either placebo or the highest dose of methylphenidate. The healthy controls were screened using structured diagnostic interview (K-SADS-E; Orvaschel, H. & Puig-Antich, J., The schedule for affective disorders and schizophrenia for school-age children-epidemiologic version (Kiddie-SADS-E), University of Pittsburgh, Pittsburgh, Pa., 1987), were free of any major psychiatric disorder, and had no more than 3 out of 9 possible symptoms of inattention or hyperactivity-impulsivity by DSM-IV criteria. Children with ADHD were included if they met criteria for ADHD on structured diagnostic interview, and had at least 6 of 9 symptoms of inattention or hyperactivity-impulsivity. Children with ADHD took part in a triple blind (parent, child, rater), randomized, placebo-controlled study of effects of methylphenidate (0, 0.5, 0.8, 1.5 mg/kg in divided dose) on activity, attention, and fMRI. Children with ADHD were treated continuously for one week with placebo or a specific dose of methylphenidate and at the end of the week were tested for drug efficacy using objective measures of attention and activity and fMRI (See Methods) within 1-3 hours of their afternoon dose. The time between dose and testing was held constant for each subject throughout the four treatment conditions. Activity and attention were evaluated in unmedicated healthy controls using the same procedure as children with ADHD, and fMRI followed within the same time frame.

T2 relaxometry, a novel fMRI procedure, was used to derive steady state blood flow measures and to test for enduring medication effects. Although conventional Blood Oxygenation Level Dependent (BOLD) fMRI is a valuable technique for observing dynamic brain activity changes between baseline and active conditions, thus far it has failed to provide insight into possible resting or steady-state differences in regional perfusion between groups of subjects, or to delineate effects of chronic drug treatment on basal brain function. T2 relaxometry, like BOLD, hinges on the paramagnetic properties of deoxyhemoglobin. However, the mismatch between blood flow and oxygen extraction that occurs as an acute reaction to enhanced neuronal activity in BOLD does not persist under steady state conditions. Instead, regional blood flow is regulated to appropriately match perfusion with ongoing metabolic demand, and deoxyhemoglobin concentration becomes constant between regions in the steady-state. Therefore, regions with greater continuous activity are perfused at a greater rate, and these regions receive, over time, a greater volume of blood and a greater number of deoxyhemoglobin molecules per volume of tissue. Thus, there is an augmentation in the paramagnetic properties of the region that is detectable as a diminished T2 relaxation time.

Conventional T2-weighted images provide only a rough estimate of T2, useful for identifying areas of pathology with markedly different T2 properties, such as tumors. To calculate T2-RT with sufficient accuracy to be able to reliably perceive small (ca. 2%) differences in T2 of gray matter associated with functional changes in blood volume, we used fast echoplanner imaging to establish a signal intensity decay curve based on 32 sequential measures at different echo times. For each of the 32 images, a refocused spin echo was observed.

Highly accurate laboratory-based measures of activity and attention were obtained by having the children perform a computerized vigilance test while an infrared motion analysis system captured and recorded movements (see Methods). These findings were used to ascertain associations between regional measures of T2-RT and capacity to inhibit motor activity to low levels while attending to a monotonous but demanding task.

As expected, boys with ADHD on placebo did not sit as still as healthy controls during the attention tests. They spent more time moving (temporal scaling: F_{1,14}=9.42, P 0.008) and had less complex movement patterns (spatial scaling: F_{1,14}=9.68, P=0.008). On the continuous performance task (CPT), a measure of attention, children with ADHD were less accurate (92.0% vs. 97.1%; F_{1,14}=2.94, P=0.10), and had a more variable response latency (F_{1,14}=3.11, P<0.10), though these differences did not reach statistical significance in this limited sample.

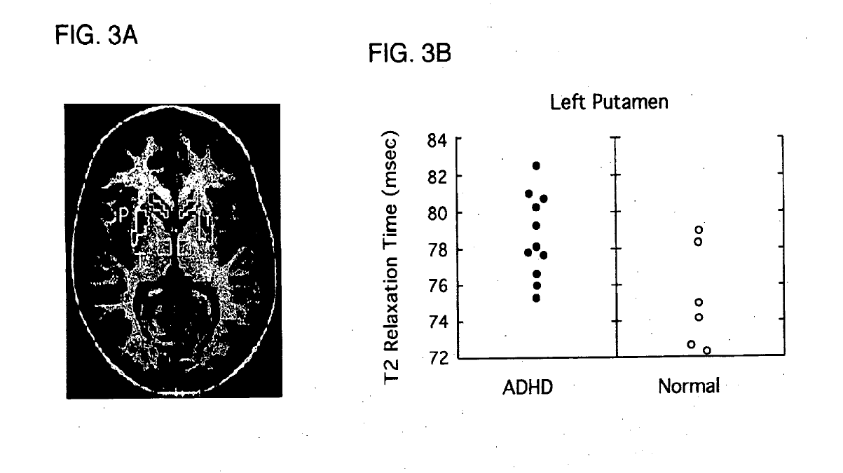

Differences in the caudate and putamen regions of children with ADHD and healthy controls, as well as the change in the T2-RT in these regions in response to methylphenidate, were also studied by imaging. The thalamus was evaluated as a contrast region in which group differences or drug effects were not expected. No significant differences emerged between ADHD children on placebo and healthy controls in bilateral T2-RT measures for the caudate nucleus (F_{1,14}=2.80, P=0.12). In contrast, ADHD children and controls differed markedly in bilateral putamen T2-RT measures (77.9±1.1 msec vs. 76.1±1.1 msec; F_{1,14}=9.40, P=0.008). On average, T2-RT was 3.1% higher in ADHD children than in controls in the left putamen (F_{1,14}=14.5, P=0.002; FIG. 3B) and 1.6% higher in the right (F_{1,14}=2.62, P=0.13).

For healthy controls and ADHD children on placebo, there were marked and significant correlations between motor activity and T2-RT for the putamen bilaterally, but not for caudate or thalamus (Table 1A). Temporal scaling and average time spent immobile, two measures of activity-inactivity, correlated −0.752 (P<0.001) and −0.730 (P <0.001), respectively with T2-RT in putamen. The complexity of the movement pattern also correlated with T2-RT in putamen (rₛ=0.630, P<0.01). Similarly, in unilateral analyses, all three motor activity measures correlated with T2 measures for both right and left putamen (Table 1A).

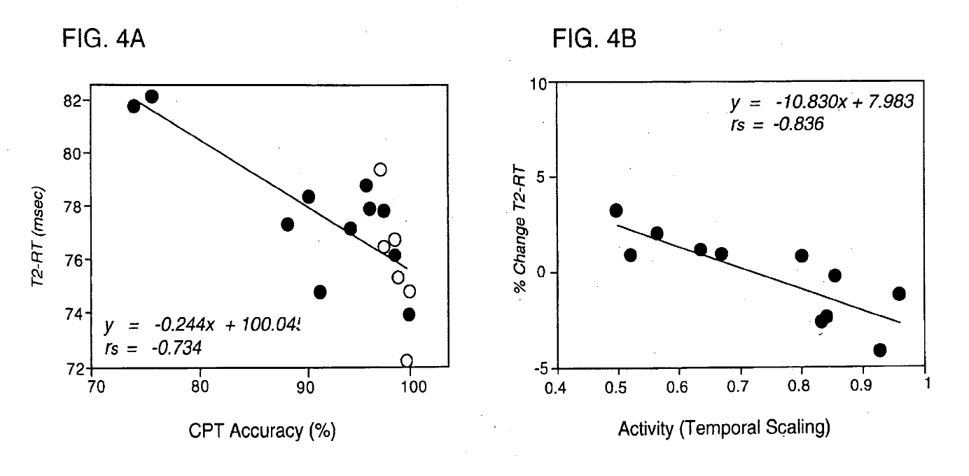

There were also robust correlations between measures of CPT performance and T2-RT in the putamen bilaterally (Table 1B). Accuracy on the CPT correlated −0.807 (P<0.0001) with T2-RT, while variability (S.D.) in response latency correlated 0.652 (P<0.005). These associations were observed in both right and left putamen (Table 1B, FIG. 4A). In addition, there was also a significant association between accuracy on the CPT task and T2-RT for right, but not left, thalamus. As indicated in FIG. 4A, there is a significant inverse linear correlation between accuracy and T2 relaxation time (higher levels of T2-RT indicate lower perfusion).

Methylphenidate exerted robust effects on attention, enhancing performance accuracy (F_{1,10}=5.98, P<0.05) and reducing response variability (S.D.) from 242 to 149 msec (F_{1,10}=14.5, P<0.005). Methylphenidate also exerted significant effects on activity, producing a 126% increase in time spent immobile (F_{1,10}=5.47, P<0.05), and increasing the complexity of the movement pattern (F_{1,10}=5.73, P<0.05). However, drug effects on activity were strongly dependent on the subject's unmedicated activity level. For instance, spatial complexity increased 52.6% in the 6 subjects who were objectively hyperactive (at least 25% more active than normal controls) on placebo (F_{1,5}=13.16, P<0.02), but was unaffected (<8% increase) in the 5 ADHD children who were not (p>0.6).

T2-RT in both right and left putamen were significantly altered by ongoing treatment with methylphenidate (ANCOVA: F_{1,9}=12.81, P=0.006), although the response was strongly tied to the subject's unmedicated activity state (Drug×temporal scaling covariant F_{1,9}=11.09, P=0.008; FIG. 4B). Methylphenidate failed to exert significant effects on T2-RT in thalamus (F₁₉=0.13, P>0.7). A trend-level difference was observed in the right caudate (F_{1,9}=3.85 P=0.08).

Overall, as higher T2-RT corresponds to lower perfusion, the present findings of increased T2-RT in the putamen of children with ADHD, and the correlation between T2-RT and objective markers of disease severity, are consistent with some earlier studies. Furthermore, the present findings also suggest that a considerable proportion of the variance between subjects in degree of hyperactivity and inattention can be accounted for by T2-RT differences within the putamen alone.

In summary, boys with ADHD (n=11) had higher T2 relaxation time (T2-RT) measures in putamen bilaterally than healthy controls (n=6; P=0.008). Relaxation times correlated with the child's capacity to sit still (rₛ=−0.75, P<0.001), and his accuracy in performing a computerized attention task (rₛ=−0.81, P<0.001). Blinded, placebo-controlled daily treatment with methylphenidate significantly altered T2-RT in the putamen of children with ADHD (P=0.006), though the magnitude and direction of the effect was strongly dependent on the child's unmedicated activity state. A similar but non-significant trend was observed in the right caudate. T2-RT measures in the thalamus did not differ significantly between groups, and were not affected by methylphenidate.

### Methods

Assessment of Activity and Attention. Activity and attention data were collected as previously described (Teicher et al., J. Am. Acad. Child Adolesc. Psychiatry 35: 334-342, 1996). In brief, children sat in front of a computer and were evaluated using a simple GO/NO−GO CPT in which the subject responds to visual presentation of a target and withholds response to a non-target stimuli that appear in the center of the screen at a fixed 2 second inertial interval (Greenberg et al., Psychopharmacol. Bull. 23: 279-282, 1987). The stimuli are simple geometric shapes that can be distinguished without right/left discrimination, and are designed to allow children with dyslexia to perform as well as normal controls. Three 5-minute test sessions were recorded during a 30-minute test period while an infrared motion analysis system (Qualisys, Glastonbury, Conn.) recorded the movement of small reflective markers attached to the head, shoulder, elbow, and back of the child. The motion analysis system stored the precise vertical and horizontal position of the centroid of each marker 50 times per second to a resolution of 0.04 mm. Results were analyzed using the concept of “micro-events.” A new micro-event begins when the marker moves 1.0 millimeters or more from its most recent resting location, and is defined by its position and duration. The spatial scaling exponent is a measure of the spatial complexity of the movement path, and is calculated from the logarithmic rate of information decay at progressively lower levels of resolution. The temporal scaling exponent is a scale invariant stochastic measure of percent time active.

Values range from 0 (immobility) to 1 (incessant activity), and are calculated from the slope of the log-log relationship between the duration of micro-events and their frequency (Paulus et al., Neuropsychopharmacology 7:15-31, 1992). Software for presenting stimuli, recording activity, and analyzing results was written by M. Teicher and licensed to Cygnex Inc.

T2 Relaxometry fMRI Procedure and Relaxation Time Computations.

Children were positioned in the scanner and instructed to remain as still as possible. Images were acquired using a 1.5-T magnetic resonance scanner (Signa, General Electric Medical Systems, Milwaukee, Wis.) equipped with a whole-body, resonant gradient set capable of echo planar imaging (Advanced NMR Systems, Inc., Wilmington, Mass.), and a standard quadrature head coil for image detection. During each examination, 3 categories of images were obtained: (1) Scout images (typically T1-weighted sagittal images); (2) High resolution T1-weighted matched axial images through the ten planes for which maps of T2 were generated; and (3) 32 spin echo, echoplanar image sets, with TE incremented by 4 msec in each consecutive image set (e.g., TE (1)=32 msec, TE (2)=36 msec, . . . TE (32)=160 msec) through the same ten axial planes (TR=10 sec, Slice thickness=7 mm with a 3 mm skip, in-plane resolution=3.125 mm×3.125 mm, FOV=200 mm). The 32 TE-stepped images were then transferred to an off-line workstation and corrected for in plane motion using a modification of the DART image registration algorithm (Maas et al., Magn. Reson. Med. 37:131-139, 1997). The value of T2-RT was then estimated on a pixel-wise basis by linear regression of the signal intensity S(x,y,n) assuming an exponential decay of S(x,y,n) with time constant T2-RT(x,y), such that ln S(x,y,TE(n))=ln S(x,y,TE=0)−(TE(n)/T2−RT(x,y)), where (x,y) is the pixel position and TE(n) is the spin-echo time corresponding to the nth image of the series.

Calculations of regional T2-RT were made for left and right anterior caudate, putamen, and thalamus (as a contrast region) using anatomic boundaries observed in T1 weighted images and conservatively circumscribed to avoid encroaching into ventricular space (see FIG. 3A for regions of interest). Delineation of regions and analysis of imaging data was performed on coded images, and the responsible researcher was blind to the identity, diagnosis, or treatment condition of the subject. T2-RT was calculated from the median value of all the designated pixels, as the median provides a regional estimate less susceptible to contamination by spurious values from bordering white matter and cerebrospinal fluid regions than the mean.

The intrinsic reliability of the T2-RT measure was determined using a within subject procedure with head repositioning when necessary. There was a lag between end of the first session and start of the second session of ca. 5 minutes. Based on 8 within-session comparisons with normal adult volunteers we observed a correlation of 0.942, and an average mean value difference of −0.17% for T2-RT of the putamen.

Statistical Analyses. Differences between groups was assessed using ANCOVA with age as a covariate. Although the groups did not differ significantly in age, the behavioral and fMRI measures showed age-dependent changes, and ANCOVA minimized this component of the error variance. Correlations were calculated using Spearman Rank-Order test. Differences between behavioral and fMRI measures of ADHD subjects on methylphenidate vs. placebo were assessed using repeated measure ANCOVA with placebo activity (temporal scaling) as a covariate. This was crucial in the analysis, as methylphenidate effects are strongly rate-dependent, and basal activity on placebo accounted for ca. 50% of the magnitude of the medication effect.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the appended claims.

What is claimed is:

1. A method of treating depression, obsessive-compulsive disorder (OCD), or attention deficit hyperactivity disorder (ADHD) in a mammal, said method comprising administering to said mammal a combination comprising (i) CDP-choline and (ii) an omega-3 fatty acid, wherein said combination is administered in a therapeutically effective amount.

2. The method of claim 1, wherein said CDP-choline or omega-3 fatty acid is administered in a subtherapeutically effective amount.

3. The method of claim 1, wherein said omega-3 fatty acid is eicosapentaenoic acid, docosahexaenoic acid, or α-linolenic acid.

4. The method of claim 1, wherein said omega-3 fatty acid is administered as fish oil, flaxseed oil, or microalgae.

5. The method of claim 1, wherein said method treats depression.

6. The method of claim 5, wherein said depression is unipolar depression or dysthymia.

7. The method of claim 1, wherein said method treats ADHD.

8. The method of claim 1, wherein said method treats OCD.
